(19) United States
(12) Patent Application Publication
(10) Pub. No.: US 2002/0013538 A1
(43) Pub. Date: Jan. 31, 2002

(54) Method and apparatus for health signs monitoring

(76) Inventor: David Teller, Novato, CA (US)

Correspondence Address: Ajay A. Jagtiani, Jagtiani & Associates, Democracy Square Business Center, 10379-B Democracy Lane, Fairfax, VA, 22030, US

(21) Appl. No.: 09/925,568

(22) Filed: Aug. 10, 2001

Related U.S. Application Data
(63) Continuation-in-part of application No. 09/306,907, filed on May 7, 1999.
Application No. 09/306,907 is a continuation-in-part of application No. 08/940,349, filed on Sep. 30, 1997, now abandoned.

(51) Int. Cl. A61B 5/00; G08B 21/00

(52) U.S. Cl. 600/549; 340/870.16; 128/903

(57) ABSTRACT

A method for monitoring health signs of an individual is provided including the steps of detecting at least one health sign characteristic of the individual with a sensor unit that is located proximate to the individual; producing a health signal from the sensor unit that indicates at least one health sign of the individual; communicating the health signal from the individual to a receiving unit over a wireless connection; processing the health signal to determine if an emergency condition exists; and providing an indication of an emergency condition to a destination node of a network, wherein operating electrical power is applied to the receiving unit in an initialization mode, the receiving unit determining if the receiving unit has received an identification signal from the sensor unit, and receiving a health signal only from a sensor unit having the received identification signal.

### CROSS-REFERENCE TO RELATED APPLICATIONS

This application is a continuation-in-part of U.S. patent application Ser. No. 09/306,907, filed May 7, 1999, which is a continuation-in-part of U.S. patent application Ser. No. 08/940,349, filed Sep. 30, 1997, the entire contents and disclosures of which are hereby incorporated by reference.

### BACKGROUND OF THE INVENTION

1. Field of the Invention

The present invention relates generally to monitoring the health of an individual and, more particularly, to remote monitoring of health signs.

2. Description of the Prior Art

It is known in a hospital setting to continuously monitor various health signs of a patient, such as temperature and heart rate, by utilizing relatively complicated and expensive equipment. For example, equipment routinely employed in hospital intensive care units includes temperature sensors that are thermally coupled to the skin of a patient with adhesive. One or more wires run from the patient to a display monitoring device. Such equipment may be more intrusive than desired, as it can be annoying for the patient to deal with the sensor wires that extend from the hospital bed, but the need for continuous monitoring and rapid reaction to changes in patient condition make the intrusion necessary. This is particularly the case in a hospital setting, where changes in health signs, such as temperature and heart rate, may be critical. Nevertheless, the monitoring equipment may be sufficiently intrusive that the patient's rest can be disturbed. It would be advantageous if the monitoring system employed in the hospital setting were less intrusive to the patient's comfort.

Also, monitoring equipment that transmits data by means of radio frequency (RF) signals can cause interference among multiple equipment units located near each other, as in a hospital environment. In addition, monitoring equipment should be readily usable from patient to patient, so that resources may be deployed exactly where needed and may be used by different patients.

There are situations in which continuous monitoring of vital health signs in a home setting may be desirable. However, hospital-grade equipment does not represent a viable alternative for a home environment, as the equipment is usually quite expensive. Moreover, hospital equipment is typically much more intrusive than is necessary in the home setting, where changes in conditions are typically not so critical that the intrusiveness of hospital equipment is justified. In addition, hospital equipment typically includes complicated connections to warning displays or other monitoring equipment that does not exist in the home environment. Furthermore, the general populace is quite unprepared to operate such sophisticated equipment and most homes lack any reliable patient assistance, such as an in-home caregiver, who could respond to any warning signals produced by such sophisticated equipment.

Other equipment typically used in the home environment is less intrusive and more simple to use, but is not generally suitable to the continuous monitoring of health signs. For example, thermometers are readily available for home use, but any thermometer reading must be manually taken each time an individual's temperature is needed. The same is true for heart rate, blood pressure, and other health-related information. As noted above, the continuous presence of health care assistance in the home cannot be reliably depended upon for performing such tasks. In addition, it can be difficult to properly interpret changes in health signs, or even be aware of changes over time that may indicate some form of health trouble for an individual. Finally, some monitoring systems are integrated with garments that are not easily transferred as children grow or circumstances change.

From the discussion above, it should be apparent that there is a need for convenient and minimally intrusive monitoring of health signs, with dependable monitoring of the health signs for any indication of trouble. The present invention fulfills these needs.

### SUMMARY OF THE INVENTION

It is therefore an object of the present invention to provide a health signs monitoring system that is minimally intrusive.

It is a further object to provide a health signs monitoring system that provides dependable monitoring of health signs for indications of trouble or emergency conditions.

According to a first broad aspect of the present invention, there is provided a method for monitoring health signs of an individual, including the steps of detecting at least one health sign characteristic of the individual with a sensor unit that is located proximate to the individual; producing a health signal from the sensor unit that indicates at least one health sign of the individual; communicating the health signal from the individual to a receiving unit over a wireless connection; processing the health signal to determine if an emergency condition exists; and providing an indication of an emergency condition to a destination node of a network, wherein operating electrical power is applied to the receiving unit in an initialization mode, the receiving unit determining if the receiving unit has received an identification signal from the sensor unit, and receiving a health signal only from a sensor unit having the received identification signal.

According to a second broad aspect of the invention, there is provided a method for monitoring health signs of an individual including the steps of communicating a health signal from a sensor unit on the individual to a receiving unit; and processing the health signal to determine if an emergency condition exists, wherein the sensor unit receives electrical power from a battery, battery power is determined by counting the number of health signal transmissions that have occurred since electrical power was last applied, and an indication of low battery power is provided when a predetermined number of transmissions have occurred.

According to a third broad aspect of the present invention, there is provided a method for continuously monitoring health signs of a patient over a period of time including the steps of attaching a sensor unit to the patient; producing a patient signal that indicates the current status of a patient health sign; transmitting the patient signal to a receiving unit; displaying the patient's current health sign status at the receiving unit, wherein the sensor unit receives electrical power from a battery, battery power is determined by counting the number of signal transmissions that have occurred since electrical power was last applied, and an indication of low battery power is provided when a predetermined number of transmissions have occurred.

According to a fourth broad aspect of the present invention, there is provided a system for monitoring health signs of an individual including a sensor unit for producing a health signal that indicates at least one health sign of an individual, the sensor unit being located proximate to an individual; and a receiving unit for receiving the health signal from the sensor unit over a wireless connection, the receiving unit including identification determination means for determining if the receiving unit has received an identification signal from the sensor unit; and means for allowing the receiving unit to receive the health signal from the sensor only after the receiving unit has received the identification signal from the sensor unit; and emergency condition determination means for determining if an emergency condition exists based on the health signal received from the sensor unit.

According to a fifth broad aspect of the present invention, there is provided a system for monitoring health signs of an individual including a sensor unit for producing a health signal that indicates at least one health sign of an individual, the sensor unit being located proximate to an individual; and a receiving unit for receiving the health signal from the sensor unit over a wireless connection, the receiving unit including emergency condition determination means for determining if an emergency condition exists based on the health signal received from the sensor unit; means for receiving electrical power from a battery; means for determining battery power by counting the number of health signal transmissions that have been received by the receiving unit from the sensor unit since electrical power was last supplied to the receiving unit; and means for providing an indication of low battery power when a predetermined number of health signal transmissions have occurred since electrical power was last supplied to the receiving unit.

According to a sixth broad aspect of the present invention, there is provided a system for monitoring health signs of an individual including a receiving unit for receiving a health signal from a sensor unit over a wireless connection, the receiving unit including identification determination means for determining if the receiving unit has received an identification signal from the sensor unit; and means for allowing the receiving unit to receive the health signal from the sensor unit only after the receiving unit has received the identification signal from the sensor unit; and emergency condition determination means for determining if an emergency condition exists based on the health signal received from the sensor unit.

According to a seventh broad aspect of the present invention, there is provided a system for monitoring health signs of an individual including a receiving unit for receiving a health signal from a sensor unit over a wireless connection, the receiving unit including emergency condition determination means for determining if an emergency condition exists based on the health signal received from the sensor unit; means for receiving electrical power from a battery; means for determining battery power by counting the number of health signal transmissions that have been received by the receiving unit from the sensor unit since electrical power was last supplied to the receiving unit; and means for providing an indication of low battery power when a predetermined number of health signal transmissions have occurred since electrical power was last supplied to the receiving unit.

According to an eighth broad aspect of the present invention, there is provided a system for monitoring health signs of an individual including a first sensor unit for producing a health signal that indicates at least one health sign of an individual, the sensor unit being located proximate to an individual; and a receiving unit for receiving the health signal from the sensor unit over a wireless connection, the receiving unit including means for determining the format of the health signal from the first sensor unit; emergency condition determination means for determining if an emergency condition exists based on the health signal received from the sensor unit.

According to a ninth broad aspect of the present invention, there is provided a method for monitoring health signs of an individual including detecting at least one health sign characteristic of the individual with a sensor unit that is located proximate to the individual; producing a health signal from the sensor unit that indicates at least one health sign of the individual; communicating the health signal from the individual to a receiving unit over a wireless connection; extracting at least one health factor from an independent data source; processing the health signal and the extracted health factor to determine if an emergency condition exists.

Other features and advantages of the present invention should be apparent from the following description of the preferred embodiment, which illustrates, by way of example, the principles of the invention.

### BRIEF DESCRIPTION OF THE DRAWINGS

FIG. 1 is a schematic diagram of a health signs monitoring system constructed in accordance with the present invention;

FIG. 2 is a representation of the sensor unit illustrated in FIG. 1;

FIG. 3 is a block diagram of the sensor unit illustrated in FIG. 2;

FIG. 4 is a block diagram of the display unit illustrated in FIG. 1;

FIG. 5 is a block diagram of the computer port unit illustrated in FIG. 1;

FIG. 6 is a block diagram of the computer illustrated in FIG. 1;

FIG. 7 is a block diagram of the network interface unit illustrated in FIG. 1;

FIG. 8 is a flow diagram showing the operating steps performed by the health signs monitoring system of FIG. 1;

FIG. 9 is a flow diagram that shows the operating steps executed in performing system initialization and registration of the transmitting unit with the receiving apparatus;

FIG. 10 is a schematic diagram of a harness embodiment of the health signs monitoring system constructed in accordance with the present invention; and

FIG. 11 is a perspective view of the monitoring harness illustrated in FIG. 1.

### DETAILED DESCRIPTION OF THE PREFERRED EMBODIMENT

It is advantageous to define several terms before describing the invention. It should be appreciated that the following definitions are used throughout this application.

Definitions

Where the definition of terms departs from the commonly used meaning of the term, applicant intends to utilize the definitions provided below, unless specifically indicated.

For the purposes of the present invention, the term “health signs” refers to an measurable or detectable signal or indicator of a health status or characteristic of an individual.

For the purposes of the present invention, the term “emergency condition” refers generally to a potentially life or health damaging or threatening condition that causes or initiates a response by the health signs monitoring system. The emergency condition may not actually be life or health damaging or threatening, but rather may simply be indicative of such a life or health concern that causes or initiates a response by the health signs monitoring system.

For the purposes of the present invention, the term “proximal” refers to a position near or adjacent to the point of reference, and is used to distinguish from the term “distal” which refers to a position away from the point of reference. For example, a sensor unit located proximal to an individual is located on the individual or near the individual.

For the purposes of the present invention, the term “household appliance” generally refers to a device or apparatus that may be contained within an individual's house. As used herein, the term “household appliance” refers to a device or apparatus, whether of a medical nature or not, that may be controlled from a remote location to bring about a particular action or result. The term “household appliance” is used herein to refer to a device or apparatus used to assist an individual or patient experiencing a distress or emergency condition. One example of a household appliance is a blood pressure cuff. In the context of this invention, individuals may be monitored for a variety of health signs, such as blood pressure. In response to various physiological signs, such as elevated heart rate or increased ambient temperature, the system of the present invention may send an appropriate signal to cause a blood pressure cuff coupled to an individual to inflate and measure the individual's current blood pressure. Another example of such a household appliance is an air conditioning unit or a fan. According to the present invention, a health signs monitoring system may determine that an individual is suffering from some health distress because the ambient temperature is too high. In this situation, the system may activate the air conditioning unit or reset the temperature level of the air conditioning unit to cool the room.

For the purposes of the present invention, the term “health factor” refers to data that may affect the health of an individual and, in addition, may assist in explaining a physiological state of such an individual. An example of a health factor is the external or outdoor air temperature in the vicinity of the individual. When an elevated blood pressure is sensed by the present invention, an elevated outdoor air temperature, if present, may assist in explaining the situation. Another example of a health factor is the pollen count in the vicinity of the individual. A pollen count of a certain level may explain or assist in explaining why the system of the present invention has sensed that a particular individual is having difficulty breathing.

For the purposes of the present invention, the term “independent data source” refers to a source of data that is not dependent on the particular health or physiologic state of an individual. Examples of independent sources are the Internet and stored databases. Such independent sources may provide ready sources for health factors that may be used to assist in explaining a particular physiologic state of an individual.

Description

In accordance with the present invention, a system for monitoring health signs of an individual is provided that detects at least one health sign characteristic of the individual with a health signs sensor unit that is located proximate the individual, produces a health signal from the health signs sensor that indicates at least one health sign of the individual, communicates the health signal from the individual to a receiving apparatus over a wireless connection, provides the communicated health signal to a network and processes the provided signal at a destination node of the network to indicate if an emergency condition exists. The system preferably sends the health signal to a receiving apparatus over a wireless transmission link and to a computer network, and processes the signal to indicate if an emergency condition exists. The components associated with the wireless transmission link may be sufficiently small and lightweight that the components may be clipped to a patient's garment or on a harness, which may be worn by the patient without undue discomfort. Unlike systems that connect a patient with wires to a monitor that then transmits information in a wireless link, the present invention needs no wired connection to any monitoring apparatus. In this way, health signs of an individual may be dependably monitored in a relatively convenient and minimally intrusive manner for any indication of trouble. This monitoring technique may be readily applied in both the hospital environment and in the home setting.

In one aspect of the invention, the sensor unit may process the health signs signal before transmitting to the receiving apparatus so as to reduce the amount of data that is transmitted over the wireless link. The remote node of the computer network may comprise a multimedia server, such as a conventional personal computer or similar device with audio and video capabilities. If desired, the computer network may comprise a network such as the Internet, and/or the multimedia server may be placed in a remote location. The multimedia server may then communicate with other remote locations, such as health care monitoring facilitates, to transmit the data generated by the health signs sensor, or may control a device or appliance.

In another aspect of the invention, each sensor unit may be encoded with a unique identification number that may be communicated whenever it transmits a signal, and the receiving apparatus registers the first identification number it receives when power is first applied to the receiving apparatus. The receiving apparatus may thereafter ignore health signs messages it detects from sensor units other than the one with which it is registered. This provides a convenient way to ensure that multiple sensor units may be used in close proximity without interference or interruptions in service.

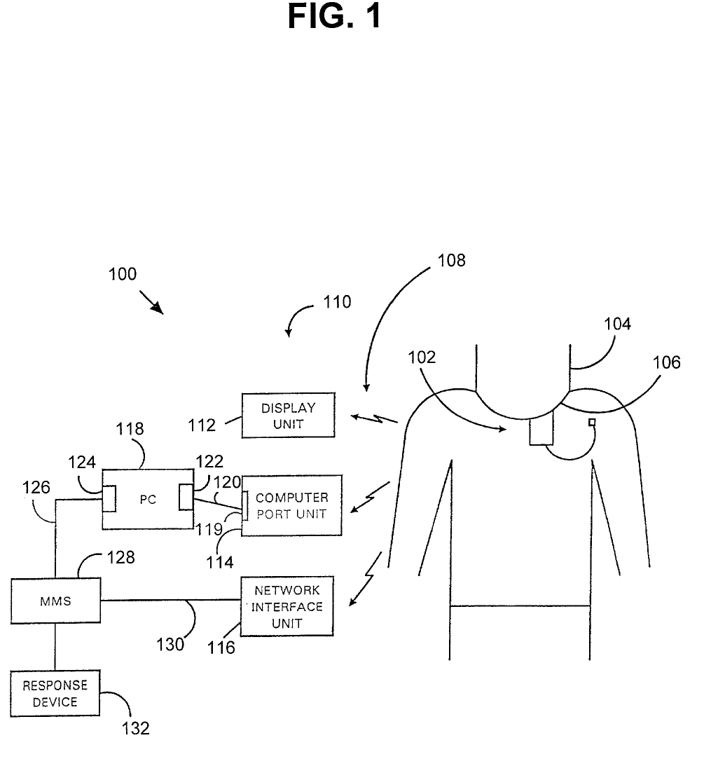

FIG. 1 illustrates a health signs monitoring system 100 constructed in accordance with the present invention. System 100 includes a sensor unit 102 that is worn by a patient 104 whose health signs are to be monitored. Sensor unit 102 is sufficiently small and light that it may be clipped to a garment 106 and worn by patient 104. In this way, sensor unit 102 will only minimally intrude on patient comfort and mobility. Signals emit from sensor unit 102 sent by means of a wireless transmission 108 to a receiving apparatus 110. Receiving apparatus 110 receives the health signs information and processes the information so that a health care provider, or other assistance, may determine if a response to the patient condition is required. In this way, system 100 provides continuous health signs monitoring with minimal intrusion to the patient's lifestyle.

Receiving apparatus 110 may comprise any one of a number of alternative receiving devices, including, for example, a display unit 112, a computer port unit 114, or a network interface unit 116. As described further below, a display unit 112 communicates the health signs information to a health care provider, such as a nurse or other assistant. Computer port unit 114 communicates health signs information to a computer 118, such as from a data port 119 through an appropriate cable 120 to a serial port or parallel port connection 122 in computer 118. Computer 118 includes a network interface 124 to connect computer 118 with a network link 126 to a multimedia server (MMS) 128. MMS 128 may comprise a server on a wide area network, such as the Internet, or on a local intranet. Network interface unit 116 communicates health signs information over a network link 130, such as an Internet connection, to MMS 128. MMS 128 may be connected to one or more response devices 132, including a pager, telephone, facsimile machine, computer, or a device or appliance located with a health care provider or in the household of the patient being monitored.

The health signs that may be monitored using a system according to the present invention may include a wide variety of patient characteristics that indicate the health of an individual, such as temperature, heart rate, blood pressure, respiration, blood oxygen, moisture, muscle response, patient body position, etc.

Wireless transmissions for use in the present invention may involve a radio frequency (RF) signal or other wireless technologies, such as the “Personal Area Network ” technique available from International Business Machines Corporation (IBM Corporation). Wireless transmission eliminates hard-wired connections from the sensor unit to a base station receiver and frees the patient from negotiating bedside wires that tend to get entangled. In the case of RF technology, the sensor unit preferably includes a small transmitter.

A display unit, or a computer port unit of the present invention may serve as a means to communicate health signs information to a health care provider, or other assistance, to determine if an emergency condition exists. In addition, various combinations of such units may be integrated into a single receiving apparatus to provide a multi-function receiving apparatus, if desired.

The network link of the present invention may be, for example, a computer connection to a telephone line, optic cable, high-speed wire, or other communications link.

Sensor Unit

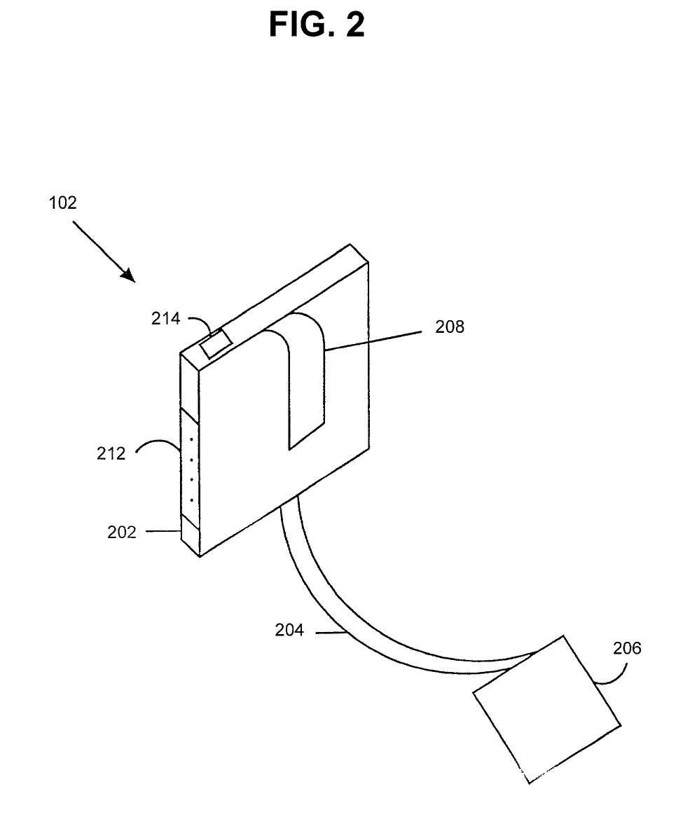

FIG. 2 is a perspective view of sensor unit 102 illustrated in FIG. 1, and shows that sensor unit 102 comprises a small transmitting card 202 from which extends a connecting wire 204 that ends in a sensor pad 206. Sensor pad 206 may be attached to a location on a patient that permits optimal placement for sensing patient health signs. The location depends on the design of sensor unit 102 and the health signs being measured. As described further below, a communications port 212 is optionally provided to permit interfacing transmitting card 202 with receiving apparatus 110 (FIG. 1), if desired. A battery life indicator 214 may be provided along the top edge of the card to indicate battery lifetime, as described further below.

Transmitting card 202 is preferably no larger than a typical pager or matchbook. Transmitting card 202 may be attached to a garment worn by a patient with a clip 208 that holds the garment between the clip and backside 210 of transmitting card 202. The small size of transmitting card 202 and clip 208 permits attachment at the collar of a shirt, for example, so as to minimize intrusion into a patient's comfort and freedom of movement. Connecting wire 204 may be sized to provide desired flexibility in the placement of sensor pad 206. If desired, transmitting card 202 may have a clip 208 or other attachment device that produces an alarm signal if transmitting card 202 is removed from the garment.

The sensor pads of the present invention may be attached to a patient's skin using removable adhesives known to those skilled in the art. Alternatively, sensor pads of the present invention may be attached with cuffs or bands that ensure optimal placement. For example, sensor pads of the present invention may comprise a cuff that wraps around an arm or wrist to provide a variety of health signs signals, such as blood pressure, heart rate, respiration rate, oximetry, body temperature, etc. In particular embodiments of the present invention, the sensor pad also may comprise a saliva analysis pad lodged in a patient's mouth.

Connecting wires of the present invention may be any suitable thickness or length. A wire of approximately six inches in length has been found sufficient for optimal placement of a sensor pad with a transmitting card worn at a patient's collar.

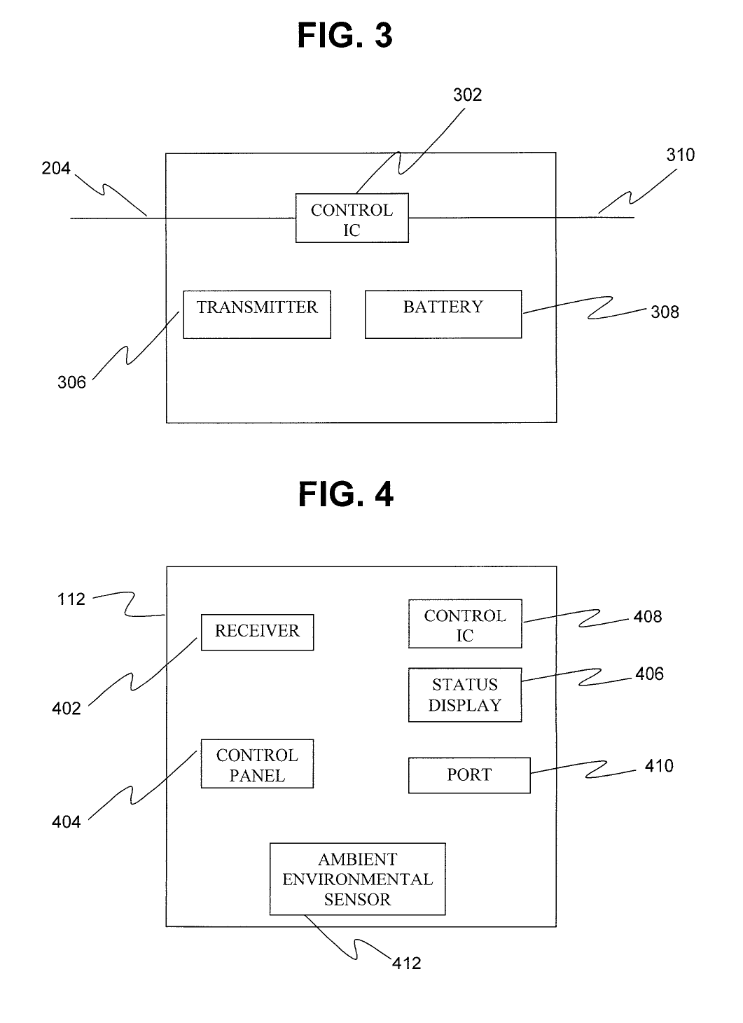

FIG. 3 is a block diagram of sensor unit 102, showing the primary functional components. A control integrated circuit (IC) 302 receives the health signs sensor signal from connecting wire 204. Control IC 302 performs processing or signal conditioning to process the signal and produce data before providing the health signs data to a transmitter 306. Sensor unit 102 may process the health signs signal before transmitting to the receiving apparatus so as to reduce the amount of data that is transmitted over the wireless link. Transmitter 306 continuously generates a wireless signal by which the health signs data may be provided to receiving apparatus 110, shown in FIG. 1.

As an alternative to wireless transmission of the health signs data to a receiving apparatus 110, a wired link may be provided through communications port 212, which may be connected to control IC 302. Communications port 212 receives a multi-pin connector (not shown) for a wire cable 310 that is coupled to receiving apparatus 110, and thereby permits control IC 302 to transmit the health signs data over a wired link.

Power for operation of sensor unit 102 is provided by a battery 308 that is preferably small and with long service life. Because of the health monitoring function of system 100, it is critical that sensor unit 102 have an able and ready source of electrical power. Typically, such assurance may be provided by battery monitoring circuitry, with a battery charge display. According to one aspect of the present invention, a battery monitoring feature is provided that eliminates the need for battery charge circuitry to detect and display remaining battery life.

In accordance with the present invention, system 100 determines remaining battery life by programming executed by control IC 302 that counts the number of signal transmissions from sensor unit 102 to receiving apparatus 110 (FIG. 1). It should be noted that, in normal operation, sensor unit 102 continuously transmits health signs signals to receiving apparatus 110. With the frequency of health signs transmission known (for example, one transmission every sixty seconds), and with the message length of each transmission known, it is relatively simple to determine the expected number of signal transmissions in a battery lifetime. Control IC 302 keeps this lifetime count, and decrements the expected number of message transmissions in the remaining life of battery 308. As soon as the lifetime count of remaining transmissions reaches a predetermined point, such as at zero transmissions left in the count, battery 308 is declared dead. Battery life indicator 214 may be, for example, a bright LED that glows steadily so long as time remains in the battery lifetime count. Once the lifetime count has been reached, control IC 302 extinguishes the indicator light, even if there is still sufficient battery power to light the LED. In this way, battery lifetime may be indicated without relatively expensive battery charge circuitry.

Transmitters of the present invention may be wireless and may make use of conventional radio frequency (RF) transmission techniques, which are well known to those skilled in the art. Alternatively, transmitters of the present invention may use Personal Area Network technology that utilizes low-voltage of a human body to electrically transfer information from the body of an individual through physical contact or proximity with a receiving body. Personal Area Network technology eliminates the need for electromagnetic transmission over large distances. The health signs monitoring system with Personal Area Network technology thereby is not affected by most RF energy, and will not interfere with the operation of RF-sensitive equipment. The transmission distance of the Personal Area Network technology is somewhat limited, so if the Personal Area Network technology is used with a sensor unit of the present invention, then a patient covering, such as a blanket, may act as a receiving unit or antenna for the Personal Area Network transmitter.

Transmitters of the present invention may serve both a transmitting and a receiving function, thus serving as transceivers. Such a transceiver provides the benefit of allowing a signal sent by a remote location to be received by the transceiver to affect a change at the sensor unit or to affect a change in the current state of treatment, analysis, measurement, etc. For example, a sensor may detect a change in a particular health signs characteristic, such as body temperature or heart rate, and when analyzed by a computer at a remote location, the computer may send a signal that will affect control of a household appliance. For example, a blood pressure cuff attached to the individual may be inflated to measure the current blood pressure of the individual. The system may also establish periods or intervals in which further measurements may be taken until a predetermined stable level has been reached. As envisioned, a physician participating in the system that comprises the current invention may observe a condition in the patient's vital signs that requires further data to accurately diagnose or treat. The physician can remotely command the system to send a signal to take a particular measurement in order to have the data required.

Suitable batteries for use in the present invention include camera batteries, watch batteries, and other batteries with long service life.

Display Unit

As shown in FIG. 1, receiving apparatus 110 may receive a health signs signal from sensor unit 102 at display unit 112. FIG. 4 shows details of display unit 112.

FIG. 4 shows that display unit 112 includes a receiver 402 that may be adapted to receive transmissions of the type sent by transmitter 306 (see FIG. 3). For example, if transmitter 306 produces radio frequency (RF) transmissions, then receiver 402 may be a conventional RF receiver. Display unit 112 also includes a control panel 404 and a status display 406, which provide a control interface through which a user provides commands and views system operating indications.

Display unit 112 continuously receives health signs data from sensor unit 102, but may store that data for processing. That is, control IC chip 408 may be programmed so that health signs data is stored in memory that is part of IC chip 408 and then is processed at regular intervals. As an example, IC chip 408 may be programmed so that health signs information sent to display unit 112 is stored in IC chip 408 memory every ten minutes, to provide a long-term record of health signs information.

In addition, as an alternative to wireless reception of the health signs data, a wired link may be provided through port 410, which may be connected to communications port 212 of sensor unit 102 (FIG. 2). Internal to display unit 112 of FIG. 4, port 410 provides a signal to control IC chip 408, and thereby permits control IC chip 408 to receive the health signs data over a wired link.

If desired, display unit 112 may incorporate an ambient environmental sensor 412 that provides an ambient environmental signal with which to perform attenuation and adjudication of the health signs signal from sensor unit 102. For example, environmental sensor 412 may comprise a microphone to subtract ambient noise from a sensor unit microphone signal, or may comprise a temperature probe to compensate the sensor unit signal for ambient temperature extremes that may otherwise provide an inaccurate or misleading signal.

Display units of the present invention may be used to display the current health signs information, such as current patient temperature, and, if desired, may also display other useful information, such as clock time or desired health signs signal values. If desired, display units of the present invention may store all display data in memory, including clock time of recording. In addition, display units of the present invention may be made to show selected messages upon the occurrence of particular health signs signals, such as a treatment for the emergency condition. For example, if a high patient temperature is indicated, then the displayed message may comprise an advertisement for a fever relief medicine. Warning messages also may be displayed.

Display units of the present invention may be designed to be easily transported to any room in which it is needed and to be of minimum size. As a result, display units of the present invention operate under control of a control IC chip. Thus, such display units do not require a complicated operating system or other peripheral resources that would increase the size and power requirements of the display unit. This ensures maximum transportability and flexibility of the system, and reduces costs.

Long-term records of health signs information may be analyzed to detect trends over time that may not be apparent over a more frequent recording schedule, or that may be tedious to review. This accommodates analysis of temperature data, which may be affected by circadian rhythms or other factors. Thus, a temperature fluctuation that may otherwise indicate an alarm will instead be determined to be a result of normal circadian fluctuation. The data recording feature of the present invention also provides data storage that is not readily accessible by unauthorized persons.

Health signs information collected over time and analyzed to determine trends may also be combined with and/or compared to data from external sources, such as stored databases and the Internet. For example, a sensor may measure blood pressure of an individual and the blood pressure data may be analyzed over time. As an additional item of information, the system computer may be connected to the Internet to extract a measurement of the environmental temperature, barometric pressure, humidity, etc. to develop an analysis that not only reflects the individual body conditions, but also takes into account environmental factors. These additional items of information are available from a variety of sources without necessitating adding additional sensors or monitors to the present system. The sources may include Internet databases, Physicians Personal Computers, Statistical databases, Genetic databases, Insurance Company Billing Databases, etc.

An additional example would be to correlate the daily pollen count in a particular area with an individual's asthma condition. At a certain level, the daily pollen count may give rise to automatic medication dispensing or administration if such a threshold level has been reached. Until such correlation has been established and accepted, the system could take more frequent measurements of environmental and patient health data during a period where an influencing environmental condition, such as pollen count, is at such a level as to warrant such increased frequency of measurement. For instance if the Internet database on pollen count (tracked by others) indicates a high pollen count for Northern California, including the zip code in which patient resides, then the amount of medication that needs to be taken may be upped by the software analysis engine and this info would be communicated to the patient via phone, email or fax.

As an alternative to using a wireless transmission means such as RF signals, a receiver of the present invention may use Personal Area Network technology that utilizes low-voltage of a human body to electrically transfer information from the body of an individual through physical contact or proximity with a receiving body. The transmission distance of the Personal Area Network technology is somewhat limited, so if the Personal Area Network is used with a sensor unit, then a patient covering such as a blanket may act as a receiving unit for the Personal Area Network transmitter.

A receiver of the present invention may also be configured as a receiver for radio frequency identification (RFID) technology from Texas Instruments Incorporated, called “TIRIS”. Those skilled in the art will recognize that TIRIS technology is currently deployed in oil company service station payment systems, which include a very low power transmitter that is passed adjacent a compatible receiving sensor to download identification data for payment. In the health monitoring system described herein, such technology may be used to download identification information for health care personnel, such as nurses. This information may be incorporated into the health signs information that is transmitted to a receiving apparatus. In addition, the TIRIS technology may be used to force a particular receiving apparatus identification code into the health signs information, as described in connection with the system initialization processing of the present invention. For example, when the TIRIS is touched to a TIRIS receiver, the identification code, the apparatus's unique identifier, is uploaded to the receiving apparatus.

Computer Port Unit

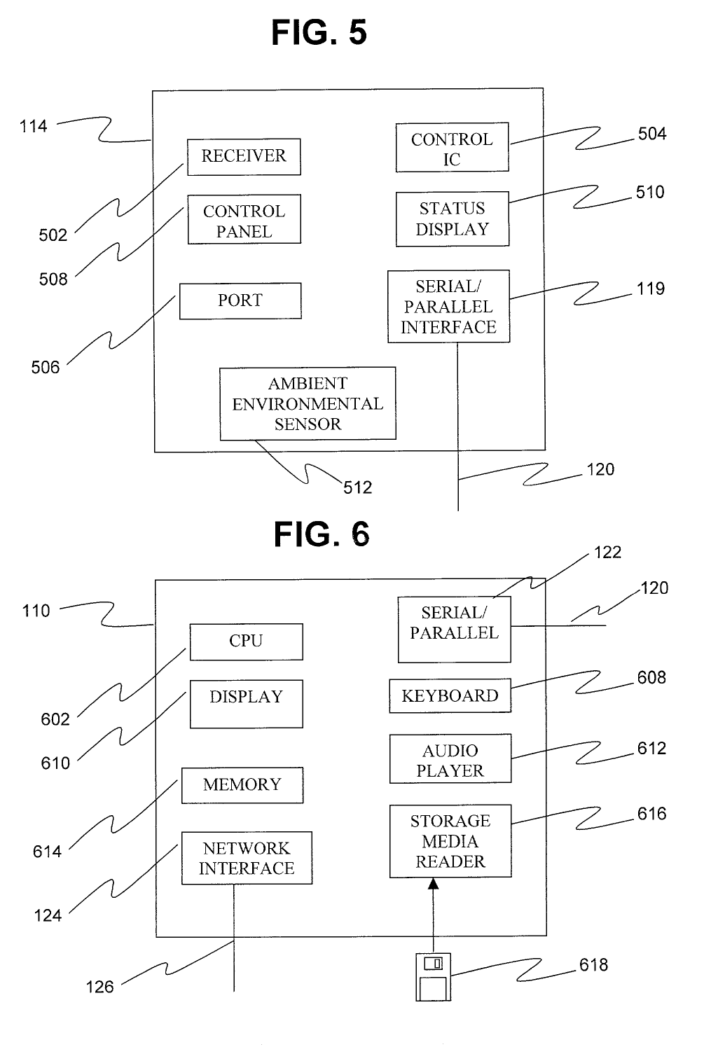

Receiving apparatus 110 may also receive a health signs signal from sensor unit 102 at a computer port unit 114. FIG. 5 shows details of computer port unit 114.

FIG. 5 shows that computer port unit 114 includes a receiver 502 that may be adapted to receive transmissions of the type sent by transmitter 306 (see FIG. 3). Computer port unit 114 operates under control of a control IC chip 504. Thus, computer port unit 114 does not require a complicated operating system or other peripheral resources that would increase the size and power requirements of computer port unit 114. This ensures maximum transportability and flexibility of the system, and also reduces cost of manufacture.

As an alternative to wireless reception of the health signs data, a wired link to a sensor unit may be provided through a port 506, which may be connected to communications port 212 of sensor unit 102 (FIG. 2). Internal to computer port unit 114 of FIG. 5, port 506 provides its signal to control IC chip 504, and thereby permits control IC chip 504 to receive the health signs data over a wired link.

Computer port unit 114 also includes a control panel 508 and a status display 510, which provide a control interface through which a user provides commands and views system operating indications.

In a similar fashion to display unit 112, computer port unit 114 may incorporate an ambient environmental sensor 512 that provides an ambient environmental signal with which to perform attenuation and adjudication of the health signs signal from sensor unit 102, such as a microphone to subtract ambient noise or a temperature probe to compensate for ambient temperature extremes. In this way, computer port unit 114 may be characterized as a receiving apparatus 110 that integrates display unit 112 with additional communications capabilities to interface with a computer 118. Moreover, system 100 may consider a combination of multiple health signs and ambient signals in determining if an emergency condition exists.

As shown in FIG. 1, computer port unit 114 interfaces with computer 118 through a cable 120 that runs from a computer data port 119 to a serial port or parallel port connection 122 at computer 118. Alternatively, cable 120 may comprise a modem line or other analog or digital means of communicating with computer 118. For example, the programming of IC chip 504 may incorporate modem functionality and automatically call and obtain a modem line to computer 118 (if so connected) at regular intervals for transfer of health signs data. Those skilled in the art will recognize that such functionality may be easily provided with IC chip 504.

Control IC chips of the present invention may be programmed so that health signs data is stored at regular intervals in memory that is part of the IC chip, such as every ten minutes, to provide a long-term record of health signs information that may be analyzed to detect trends over time that may not be apparent over a more frequent recording schedule, or that may be tedious to review. Control IC chips of the present invention may be programmed to download stored data at a predetermined time or recording interval, or such IC chips may be programmed to respond to a predetermined signal that initiates download. This feature also provides data storage that is not readily accessible by unauthorized persons.

Health signs transmissions received by computer port units of the present invention may comprise RF transmissions, in which case the receiver of the present invention is a conventional RF receiver, or may comprise Personal Area Network technology that utilizes low-voltage of a human body to electrically transfer information from the body of an individual through physical contact or proximity with a receiving body. The transmission distance of Personal Area Network technology is somewhat limited, so if a Personal Area Network is used with the sensor unit, then a patient covering, such as a blanket, may act as a receiving unit for the Personal Area Network transmitter.

Status displays of the present invention may be alphanumeric displays to show the current health signs information, such as current patient temperature, and, if desired, may also show other useful information, such as clock time or desired health signs signal values. In addition, status displays of the present invention may be made to show selected messages upon the occurrence of particular health signs signals, such as targeted advertising or warning messages.

FIG. 6 shows that computer 118 operates under control of a central processing unit (CPU) 602, such as a “Pentium” microprocessor manufactured by Intel Corporation. CPU 602 controls operation of serial/parallel port 122 to communicate with computer port unit 114 and controls operation of computer network interface 124 to communicate with MMS 128. Thus, health signs data transmitted from sensor unit 102 to computer port unit 114 may be sent to computer 118 over cable 120, and then to MMS 128.

A user controls operation of computer 118 through a keyboard 608 and display 610, which provide a means of communicating information and receiving commands from the user. Computer 118 also includes an audio player 612, which typically includes what is commonly referred to as a sound card, with appropriate sound drivers and loudspeakers. Audio player 612 permits audible warnings to be played, for example, and may optionally include a sound microphone to receive audible requests from a user or from a patient.

The operating system and applications that are executed by computer 118 may be stored in memory 614. The executed memory instructions of computer 118 implement the proper processing for communication between computer port unit 114 and MMS 128. Computer 118 also includes a storage media reader 616, such as a hard disk drive or a removable media disk drive. If storage media reader 616 is a removable media disk drive, then it typically will accept external storage media 618 such as a floppy disk or an optical (CD or DVD) disk. Such media provide a convenient means of receiving data or new programming. If storage media reader 616 also has write capabilities, then it may also provide a convenient means of downloading data or programming to other computers.

From computer 118, health signs information may be communicated to MMS unit 128, shown in FIG. 1. As noted above, MMS 128 may comprise a server on a wide area network, such as the Internet, or on a local intranet. MMS 128 may have a construction similar to that of computer 118, so that MMS 128 preferably includes at least a CPU, keyboard, display, memory, serial/parallel port, and network interface. The construction details of MMS 128 are therefore similar to those of computer 118. Additionally, MMS 128 includes whatever interfaces are necessary to support communication with response devices 132.

Displays of the present invention may comprise, for example, a typical computer display screen such as a video monitor or flat panel display.

Suitable operating systems for computers of the present invention may comprise, for example, Windows 95/98/NT/2000/XP products from Microsoft Corporation or the Macintosh Operating system by Apple Computer Corporation. Memory of the present invention may comprise any suitable memory or a mixture of semiconductor memory and other storage media in which data, commands, and program instructions may be stored.

Responsive devices of the present invention include a pager, telephone, facsimile machine, computer, or a device or appliance located at a health care provider or in the household of a patient being monitored.

Network Interface Unit

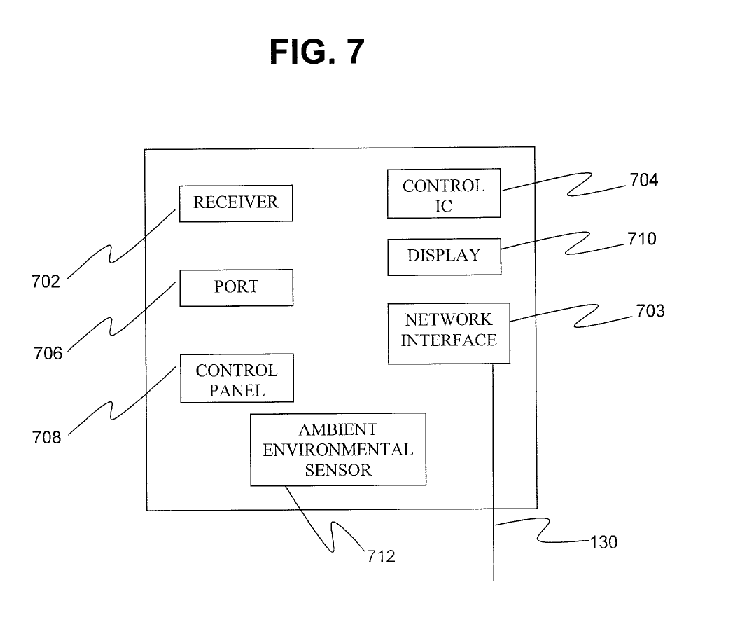

Network interface unit 116 receives signals from sensor unit 102 and provides the health signs data to MMS 128. FIG. 7 shows construction details of network interface unit 116.

FIG. 7 shows that network interface unit 116 includes a receiver 702 that is adapted to receive transmissions of the type sent by transmitter 306 (see FIG. 3). A network interface 703 permits network interface unit 116 to provide health signs data to network connection 130. Network interface unit 116 operates under control of a control IC chip 704. Thus, network interface unit 116 does not require a complicated operating system or other peripheral resources that would increase the size and power requirements of network interface unit 116. This ensures maximum transportability and flexibility of the system, and also reduces cost of manufacture.

As an alternative to wireless reception of health signs data, a wired link to sensor unit 102 may be provided through a port 706, which may be connected to communications port 212 of sensor unit 102 (FIG. 2). Internal to network interface unit 116, port 706 provides its signal to control IC chip 704, and thereby permits control IC chip 704 to receive health signs data over a wired link.

Network interface unit 116 also includes a control panel 708 and a status display 710, which provide a control interface through which a user provides commands and views system operating indications.

If desired, network interface unit 116 may incorporate an ambient environmental sensor 712 that provides an ambient environmental signal with which to perform attenuation and adjudication of the health signs signal from sensor unit 102.

Control IC chips of the present invention may be programmed so that health signs data is stored at regular intervals in memory that is part of the IC chip, such as every ten minutes, to provide a long-term record of health signs information. Such information may be analyzed to detect trends over time that may not be apparent over a more frequent recording schedule, or that may be tedious to review. Control IC chips of the present invention may be programmed to download the stored data at a predetermined time or recording interval, or the IC chip may be programmed to respond to a predetermined signal from the receiving unit that initiates download. This also provides data storage that is not readily accessible by unauthorized persons. Network interface units of the present invention may be programmed to automatically obtain a network connection and transfer health signs data at regular intervals.

Transmissions received by network interface units of the present invention may comprise RF transmissions, in which case the receiver is preferably a conventional RF receiver, or may comprise Personal Area Network technology that utilizes low-voltage of the human body to electrically transfer information from the body of an individual through physical contact or proximity with a receiving body. As noted above, the transmission distance of Personal Area Network technology is somewhat limited, so if Personal Area Network technology is used with a sensor unit of the present invention, then a patient covering, such as a blanket, may act as a receiving unit for the Personal Area Network transmitter.

Status displays of the present invention may be alphanumeric displays to show current health signs information, such as current patient temperature, and, if desired, may also show other useful information, such as clock time or desired health signs signal values. In addition, status displays of the present invention may be made to show selected messages upon the occurrence of particular health signs signals, such as targeted advertising or warning messages.

Environmental sensors of the present invention may comprise a microphone to subtract ambient noise from a sensor unit microphone signal, or may comprise a temperature probe to compensate the sensor unit signal for ambient temperature extremes that may otherwise provide an inaccurate or misleading signal.

Other Features of the Health Signs Monitoring System

Preferably, a transmitter of the present invention remains inactive until a manual switch is activated. The transmitter then preferably waits until a sensor is plugged in before beginning transmission, with only a short delay of, for example, 1 to 3 seconds.

Transmitters of the present invention may be programmed to gather data from sensors and to assemble data into packets to be transmitted. Data may be transmitted, for example, over RF frequency. Preferably, during transmission, the transmitter flashes an LED or provides an audible signal to indicate transmission. An associated receiver may be programmed to receive transmitted data, analyze and locate a desired message, and process the message, such as by displaying a message or signaling an alarm. A transmitter may also be programmed to run an initial calibration routine on each newly connected sensor. The type of sensor, the initial health sign reading, and the result of the calibration test may be assembled into a packet and transmitted to an associated receiver. The receiver may then notify the user or attendant if the calibration test was successful. Receivers of the present invention may contain a serial protocol to account for a PC or custom software, such as RS-232, user interface for data logging purposes.

According to an embodiment of the present invention, when a sensor is removed from a transmitter, a lost sensor or other message may be transmitted at short intervals such as every 5 to 20 seconds for one minute or more indicating which sensor was disconnected. After one minute or another programmed interval, the lost sensor or other message will stop and the transmitter will be allowed to return to sleep mode if no other sensors are attached thereto.

Typically, while in sleep mode, the only message that may be transmitted is a low-battery message. In instances where a low-battery message is transmitted, the receiver may sound the alarms and/or flash the display or an LED at specific intervals or until such time as the condition is resolved.

If the receiver is operating on battery power, the behavior or operating parameter of certain power-intensive features may be modified so that they do not cause significant drain on the remaining battery power. Also, in such circumstances, the receiver may flash the display or sound an alarm to alert the user or attendant.

Receivers of the present invention are preferably configured with both an audible and a visual alarm system. In particular embodiments of the present invention, the audible alarm may include a series of high-frequency pulses emitted from a speaker associated with the receiver. Preferably, the alarm may be muted and/or the sensor alarm thresholds may be adjusted at the receiver unit. In particular embodiments of the present invention, the receiver has a menu system accessible from the front panel of the receiver unit. The menu allows thresholds, alarm levels, display units, etc. to be selected, and after the values are set, to be stored in a memory, such as a non-volatile memory or similar memory, as default settings. The menu preferably has a timeout feature so that it does not remain on continuously and to avoid interference with display of sensor data. The menu also preferably has a delay before activating, thus requiring extended button contact, to prevent tampering or accidental activation of the menu. Specifically, temperature readings are preferably selectable in both ° F. or ° C.

A sensor signal may be processed by converting the sensor value into appropriate units based upon information encoded in the sensor message. For example, the conversion may result from the user of a temperature probe. A thermistor temperature probe has electrical properties such that changes in temperature result in changes in electrical resistance. The electrical properties are known and documented, and the relationship between electrical resistance and temperature can be expressed as a mathematical equation. The conversion process may involve the following: the electrical resistance is measured, and processed by the appropriate algorithm, and a temperature is the result. With varying degrees of complexity, this is how sensor input values may be converted. The converted value may be compared against high/low threshold values set by the user or attendant for a particular receiver. If the sensed value exceeds a threshold, an alarm or visual signal may be initiated until acknowledged by a user or attendant. The alarm or visual signal may be programmed to be continuous or to repeat at a predetermined interval until acted on by the user or attendant or until the sensed message returns to an acceptable range.

Operation of the Health Signs Monitoring System

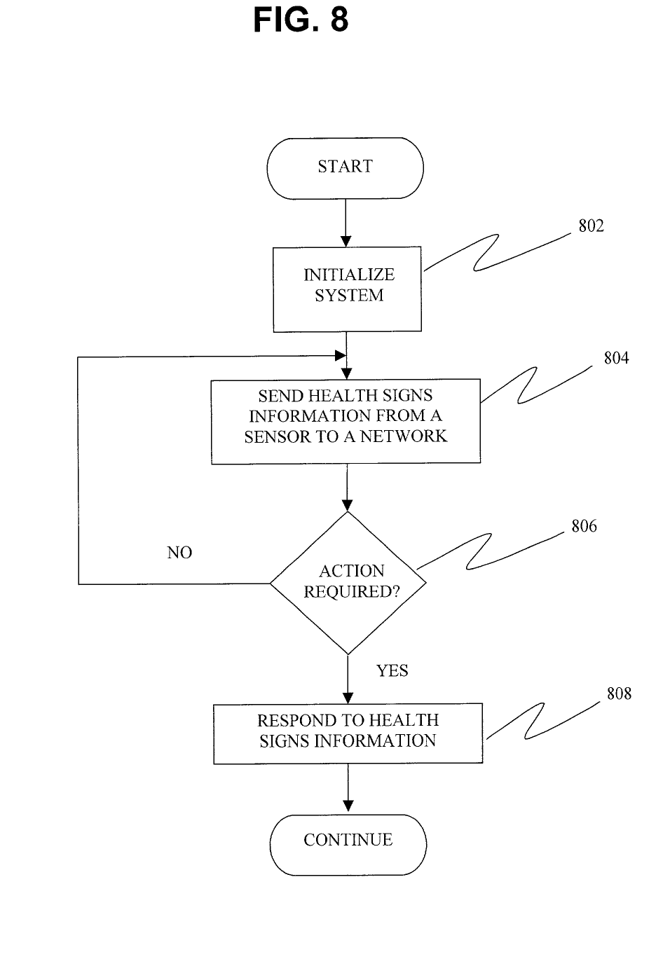

FIG. 8 is a flow diagram that shows steps performed during operation of a health signs monitoring system in accordance with the present invention. In the first step, represented by flow diagram box 802, the system is initialized as electrical power is applied. In the next step, represented by flow diagram box 804, health signs information is sent from a sensor to the network. In the next step, represented by decision box 806, the system determines whether an action should be performed in reference to the health signs information. In the next step, represented by box 808, the system responds to the health signs information.

As described further below, initialization may involve one or more sensor units making their presence known to a receiving unit. It should be understood that a health signs monitoring system in accordance with the present invention preferably includes a sensor unit and one or more receiving apparatus, which transmits health signs data to a network computer such as an MMS. Each receiving apparatus may incorporate a display. Thus, initializing the system may involve displaying health signs information and establishing communications between the sensor unit and the receiving apparatus.

The sending of information from a sensor may occur at regular time intervals, or whenever there may be a need to convey such information, such as when an emergency situation exists or when trouble is indicated. The network receiving the information may comprise a wide area network, such as the Internet, or may comprise a local network such as an intranet. The network destination for the information may be a monitoring facility staffed by persons who can appropriately respond to the information, or may simply be a computer in another room of the household where the transmitting unit is located.

Preferably, each message transmitted to a receiver includes the transmitter identification number, sensor type, sensor reading and error checks. A new message may be sent every time a sensor value changes or at predetermined intervals. If a sensor value does not change over a predetermined period of time, for example 10 minutes, a message may be transmitted to indicate that the monitored health sign did not change during that time period.

When receiving health signs information, a network may or may not respond with an action. This decision should be made because it is contemplated that monitored health signs information will typically not require any action beyond normal recording functions that may be desired. Thus, if no such extraordinary action is required, then processing returns to sending health signs information. If the health signs information indicates that some action is necessary, then processing continues, which involves responding to the health signs information.

Processing and responding to information, as well as decision making in a health signs monitoring system of the present invention may involve a variety of potential scenarios. If the health signs sensor includes a temperature sensor, for example, then the decision may involve a threshold temperature above which trouble is indicated, or a change in magnitude over a predetermined time that indicates trouble. The monitoring system may account for changes in circadian rhythm by checking time-of-day before indicating whether the body temperature of an individual is too high. That is, body temperature in the early afternoon may be expected to be higher than the body temperature in the late evening. Therefore, detected body temperatures may not trigger an alarm condition at certain times of the day, but may trigger an alarm at another time of day.

If the health signs sensor is a microphone, then the alarm decision may involve a predetermined volume level or a predetermined rate of change or magnitude of increase in volume, above which trouble is indicated. A noise level detected by the microphone that is greater than the threshold predetermined level will be interpreted as a sign of trouble. The health signs sensor may also be a combination of position or movement sensors, in which case the threshold detection may involve sensor orientation, acceleration, or other position change that indicates an undesired change. For example, a position sensor may be used to identify the presence of a possible undesireable or dangerous condition may be a “rollover” detector. It is now generally accepted that babies should sleep face up in order to reduce the possibility of Sudden Infant Death Syndrome (SIDS). A position detector may be used to identify that the baby has rolled over into an undesireable or dangerous condition and may warn of a possible undesireable or dangerous condition to prevent SIDS.

Thus, input from multiple sensors may be considered in determining if an emergency condition exists. A detected elevation in heart rate may be checked against body temperature, or vice versa, as the two are known to be interrelated. In addition to data values or magnitudes, the system may consider the magnitude of change or rate of change in a signal.

In a preferred embodiment, information may be sent from an MMS unit to the network and may involve some decision making at the receiving unit or the MMS unit. The receiving unit or MMS unit may receive temperature data or heart rate data, for example, but may not send all such received data to the network. Rather, the receiving unit or MMS unit may first process the data to make an alarm or emergency condition determination. Similarly, the receiving unit or MMS unit may receive sound or position information, from which it may determine whether an alarm should be delivered. In this way, some level of operating intelligence would be present in the receiving unit or MMS unit to decide if the health signs information sent to the network would consist of an alarm signal or a “clear” signal. Even in the absence of an alarm signal, the sensor unit typically sends health signs information to the receiving apparatus. For example, by default, health signs information may be sent from the receiving apparatus to the MMS unit at least once every half hour. An emergency condition or other sign of trouble determined at the receiving apparatus results in immediate transmission of information to the MMS. Data sent to the MMS comprises, for example, the prior half hour of health signs data. In a preferred embodiment, the MMS stores such data for each patient, so as to create and maintain a database of patient information.

In addition to operating intelligence at the receiving unit or MMS unit, there may be some level of operating intelligence at the transmitting unit. The intelligence included in the transmitting unit, for example, may be sufficient to screen or filter the temperature and heart rate data. In that circumstance, the transmitting unit may receive temperature data or heart rate data, but may not send all such data to the receiving unit. Rather, the transmitting unit itself may include operating intelligence, or programming, to check for rate of change or magnitude of reading before sending health signs information to the receiving unit. In that case, the health signs information sent from the transmitting unit to the receiving unit indicates whether an alarm condition is present and does not necessarily include raw data. As above, this conserves transmission bandwidth and reduces energy consumption. This is especially important for the transmitting unit, which depends on battery power for operation.

The response to health signs information preferably takes place after information is sent over the network by, for example, an MMS unit to a destination network node. The response, however, may involve action at the site of a transmitting unit and receiving unit. For example, health signs information may indicate that an individual's heart rate is elevated. A response to health signs information may involve controlling a home appliance such as by automatically reducing a home heating thermostat or turning on cooling equipment, or may involve contacting local paramedic or ambulance services. For example, for a patient or individual suffering from sleep apnea, the health signs monitoring system of the present invention may cause the individual's bed to shake to wake the individual to help restore their breathing. The intelligence to determine that a reduction in temperature is called for and to generate a command to make such a reduction may reside in the destination computer at a network node. Thus, an automatic response to the health signs information may include automatic adjustment of household appliances or systems. If such automatic response is desired, a receiving unit or MMS unit may be adapted to control such external devices.

Initialization of Communications and Registration of Transmitting Units

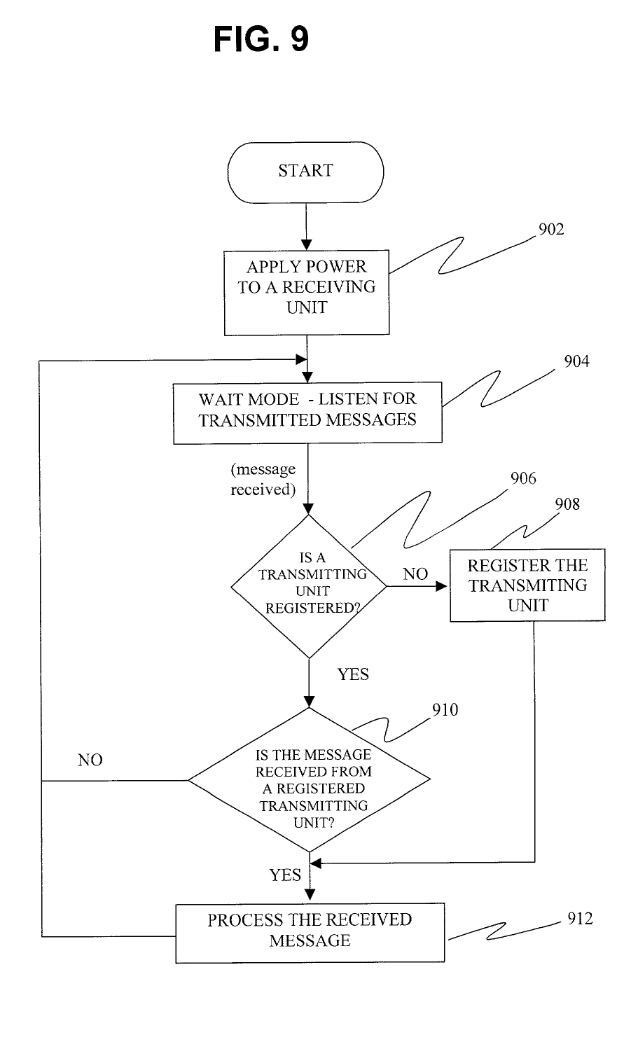

As noted above, initialization of the system involves the establishment of communication between a receiving unit and a transmitting unit. FIG. 9 shows the operating system executed in performing the initialization and subsequent operation.

Electrical power is applied to the receiving unit, represented by flow diagram box 902. The receiving unit next waits to receive a transmitted message from a transmitting unit, as indicated by flow diagram box 904. When a message is received, the receiving unit determines whether a transmitting unit is registered, shown by box 906.

If there is no transmitting unit registered, the receiving unit registers the transmitting unit, shown by box 908. After the transmitting unit is registered, any health signs information transmitted from that transmitting unit may be processed, shown by box 912. After the message is processed, the system returns to a wait mode until another message is received.

If there is a transmitting unit registered, the receiving unit determines whether the message is being received from a registered transmitter unit, shown by box 910. If the message is not being received from a registered transmitting unit, the system returns to a wait mode until another message is received. If, however, the message is being received from a registered transmitting unit, the message is processed, shown by box 912. After the message is processed, the system returns to a wait mode until another message is received.

In accordance with a preferred embodiment of the present invention, the control IC chip of each transmitting unit may be encoded with a unique identification number at the time of manufacture. Thus, the particular transmitting unit may be easily identified during its operation life. The transmitting unit identification number may be sent by the IC chip with every transmission, so that a receiving unit may immediately identify the source of a health signs information message. A receiving unit registers the first transmitting unit it identifies after electrical power is applied to the receiver. Upon each power initialization-cycle, the receiver again performs the registration function to associate itself with a particular transmitting unit. This is useful for operation in a crowded environment, where multiple transmitter receiver pairs may be in close proximity and may use the same communications channels. The identification number permits identification of appropriate transmitting units, and exclusion of messages from all others.

An initial calibration step may also be performed. A packet of information, including the transmitter identification number, may be sent to the receiver continuously or at defined intervals to establish connectivity with the receiver. Thus, when a transmitted message is received at a receiving unit, the receiving unit determines if it should process the message by first checking to determine if it has registration information from a transmitter. When a signal is received, the message is stored and decoded to obtain the transmitter identification number. The receiver compares the transmitter identification number with the number stored in the receiver's memory. The receiver utilizes the transmitter identification number to determine if the receiver should accept the message. If no transmitting unit is registered, then the receiving unit registers the transmitter that sent the message. To register the transmitting unit, the receiving unit may obtain the control IC identification number of the transmitter from the received message and store it in memory or a register of the receiving unit.

In certain instances where a new sensor must be registered, a sensor containing a transmitter or transceiver may be connected to the present system wherein the display unit is not familiar with the transmission protocol. In such situations, the computer or receiver receives a signal via the display unit that requires the computer to supplement the display unit with the protocol for understanding and displaying the new sensor data format. The computer is programmed with a lookup table that includes a certain number and variety of sensor data formats. In this manner, the display unit is simplified and does not have to maintain unnecessary sensor and transceiver protocols. A fundamental functionality of the basic communication protocol is to transmit the “type” of the registered or transmitting sensor device. The lookup table is employed to determine if the transmitting or registering device is of a currently supported type on this particular display unit. If the sensor is of a type for which there is no matching entry in the lookup table, then the software on the display unit is programmed to inform the user, connect to the Internet, download and install the new protocol specific to the newly registering device.

In a preferred embodiment of the present invention, if there is a transmitting unit registered with the system, then the control IC identification number of the transmitter should already have been stored in memory or a register of the receiving unit, and the receiving unit may therefore check to determine if the registered transmitting unit is the one that sent the received message. If the received message is from the registered transmitting unit, then the receiving unit processes the received message. This processing may comprise a variety of actions, such as the actions described above in conjunction with FIG. 8. If the received message is not from a registered transmitter, then processing continues without processing the received or detected message.

If the identification numbers do not match, the message may be discarded and the system returned to a waiting state. If no valid message has been received within a predetermined time frame, such as 5 minutes, 10 minutes, 15 minutes, etc., the receiver may be programmed to sound and alarm or display a “no signal” message. The alarm or “no signal” message may be made to be continuous or repeating at defined intervals until acted on by a user or attendant or until a valid message is received.

Alternative Embodiment: Harness Unit

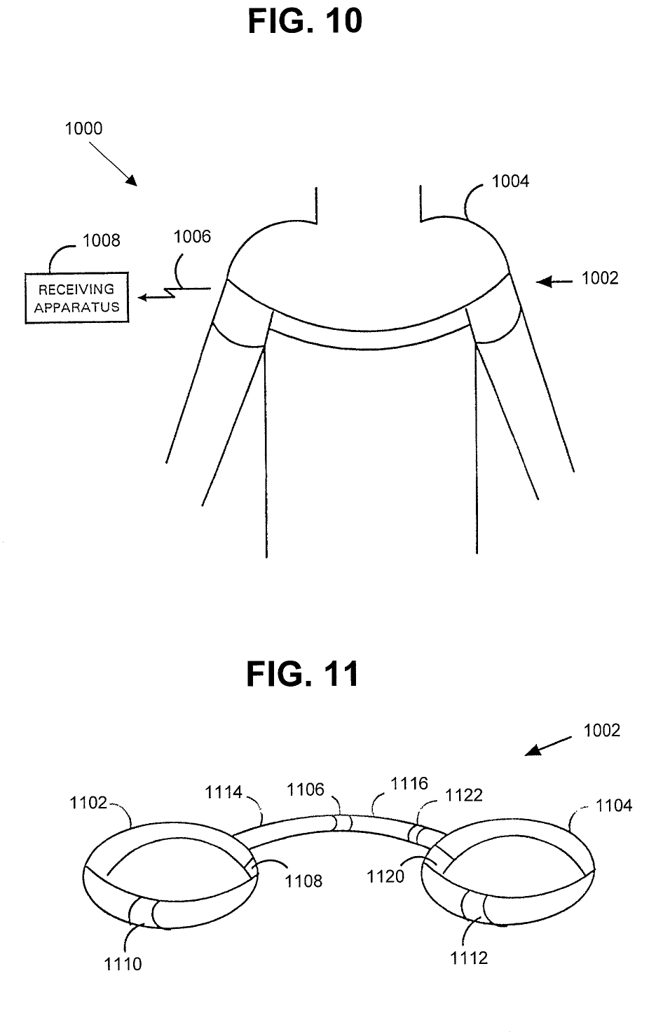

Rather than clipping sensor unit 102 to a garment, a sensor unit of the present invention may be integrated with a harness unit. FIG. 10 shows a health signs monitoring system 1000 constructed in accordance with an embodiment of the present invention. System 1000 includes a harness unit 1002 that may be worn by a patient 1004 whose health signs are to be monitored. Health signs information from harness unit 1002 may be sent by means of wireless transmission 1006 to a receiving apparatus 1008 that processes the information.

FIG. 11 shows harness unit 1002 in greater detail. In normal use, a patient's arms may be slipped into soft loops 1102, 1104 and a slip clasp 1106 may be adjusted so harness unit 1002 is comfortable and yet sufficiently snug around the patient's chest so the patient will not disturb the placement of a health sign sensor unit 1108. Sensor 1108 continuously monitors a vital function or health sign, such as temperature or heart rate. Sensor unit 1108 may be a card unit similar to sensor unit 102 described previously. Soft loops 1102, 1104 include slip clasps 1110, 1112 respectively, that may be adjusted to lengthen or shorten the soft loops to fit harness unit 1002 to the patient comfortably. All together, clasps 1106, 1110, 1112 ensure a comfortable fit of harness unit 1002 to most patients. If desired, an additional health sign sensor 1120 may be located on soft loop 1104.

Two elastic straps 1114, 1116 of harness unit 1002 may be joined by a first slip clasp 1106. The joined straps tend to remain above the upper curve of the back, just below the neck, because the straps resist stretching over the bony hump caused by the physiognomy of the upper back. This helps ensure that the entire harness unit remains in a preferred position for best operation of the health sign sensor.

If sensors 1108, 1120 are not constructed similar to sensor unit 102 (FIG. 1) with transmitters, then harness unit 1002 preferably includes a transmitting unit 1122 that receives the health signal from sensors 1108, 1120 and transmits the signal(s) to receiving apparatus 1008 (FIG. 10). Transmitting unit 1122 may be positioned on top of the individual's shoulder, or may be placed on the outside of a loop 1102, 1104 for greater comfort in wearing harness unit 1002.

As with the clip-on system, the health signs that may be monitored using a harness unit of the present invention include patient temperature, heart rate, blood pressure, respiration, oximetry, and sounds or noises in the vicinity of the patient that may indicate trouble or the need for attention.

A receiving apparatus of the present invention for use with a harness unit of the present invention may be a display unit, a computer port unit, a network interface unit, etc.

Harness units of the present invention may include multiple sensors or sensor units that produce signals that indicate the health signs that are to be monitored. In a preferred embodiment, for example, a first health sensor comprises a temperature sensitive sensor, such as a thermistor. The sensor may be attached to one of the soft loops or straps of the harness unit so that a patient properly wearing the harness unit may be able to attach the sensor so it is in continuous external contact with the patient's armpit. The sensor is thus preferably of thin, elongated construction, and may be secured at both ends to a loop with a soft, flexible connection. In this way, the harness unit and sensor may be worn in place comfortably for extended periods of time.

Sensors of the present invention may be temperature sensors, or may be a heart rate sensor, or some other health signs sensor. For example, sensors of the present invention may provide sound detection, because the presence or absence of sounds may indicate whether the patient is experiencing difficulty. A microphone that may be part of a software program or connected to a computer system with a software program that measures sound volume and characteristics. The sensors may detect the absence of breathing sounds, or the cries of a baby, or a falling patient or object, for example. Sensors of the present invention may also provide patient orientation information as a health sign. That is, the sensors may comprise simple mercury switches that indicate when a patient has fallen or rolled over. Such patient position information may be critical under some circumstances, such as with small children or the elderly.

Transmitting units of the present invention may make use of conventional radio frequency (RF) transmission techniques, which are well known to those skilled in the art. Alternatively, transmitting units may use Personal Area Network technology that utilizes low-voltage of a human body to electrically transfer information from the body of an individual. That is, the wireless transmission link may comprise Personal Area Network technology that eliminates the need for electromagnetic transmission, such as radio frequency (RF) signals. The health signs monitoring system with Personal Area Network technology thereby is not affected by most RF energy, and does not interfere with the operation of RF-sensitive equipment.

The system described above provides convenient and minimally intrusive monitoring of health signs, with dependable monitoring of health signs for indication of trouble in the home environment and in the hospital environment. In accordance with the invention, health signs of an individual may be detected with a health signs sensor unit producing health sign characteristics of the individual, which sends health signals to a receiving apparatus over a wireless connection and to a computer network. The system processes the signal at a remote node of the computer network to indicate if an emergency condition exists and thereby permits dependable monitoring of an individual's health signs in a relatively convenient and minimally intrusive manner.

The health signals that are produced from the sensor unit may indicate a variety of health signs, such as pollen count and air temperature. Health factors, that are from an independent data source, such as the Internet, stored database, medicine dispenser or invoice, are processed with the health signal to determine if an emergency condition exists. For example, independent data may come from a pill dispenser that may contain health factors of a patient prescribed medicine. In addition, independent data may come from an invoice that may contain health factors of the services rendered to treat a patient.

Other features may be added to a health signs monitoring system of the present invention without departing from the scope of the invention. For example, the analysis of health signs may comprise analysis of detected sound that is displayed in a graphical format, with the graphical representation data transmitted over a network or via modem. An emergency condition may then be triggered, as appropriate. One implementation of such an analysis feature may involve a microphone detecting heart and lung sounds, and producing a graphical display for analysis. Sound data may be stored and any anomaly may trigger an emergency condition notification. In addition to the monitoring function, the system may perform analysis of captured data on demand. For example, the system may be constructed to permit comparison of health signs data with data from a database to make a determination of an emergency condition. In particular, the system may capture image data with a video camera and the image data may be compared to stored images to determine if a rash, for example, comprises an emergency condition. Other devices may physically initiate health signs data for analysis, such as a device that may be placed on the body to prompt a reflex action from the knee or ankle. Sound data may be collected, if desired, as described above.

The present invention has been described above in terms of presently preferred embodiments so that an understanding of the present invention may be conveyed. There are, however, many configurations for client-server computer systems not specifically described herein but with which the present invention is applicable. The present invention should therefore not be seen as limited to the particular embodiments described herein, but rather, it should be understood that the present invention has wide applicability with respect to client-server computer systems generally. All modifications, variations, or equivalent arrangements that are within the scope of the attached claims should therefore be considered within the scope of the invention.

What is claimed is:

1. A method for monitoring health signs of an individual, the method comprising the steps of:
detecting at least one health sign characteristic of the individual with a sensor unit that is located proximate to the individual;
producing a health signal from the sensor unit that indicates at least one health sign of the individual;
communicating the health signal from the individual to a receiving unit over a wireless connection;
processing the health signal to determine if an emergency condition exists; and
providing an indication of an emergency condition to a destination node of a network, wherein operating electrical power is applied to the receiving unit in an initialization mode, said receiving unit determining if said receiving unit has received an identification signal from the sensor unit, and receiving a health signal only from a sensor unit having the received identification signal.

2. A method as defined in claim 1, further comprising:
halting the application of operating electrical power; and
repeating the steps of determining and receiving upon resuming the application of operating electrical power, until a next identification signal is received.

3. A method as defined in claim 1, wherein the step of processing the health signal comprises responding to an emergency condition by activating a sensor unit.

4. A method as defined in claim 1, wherein the step of processing the health signal comprises responding to an emergency condition by activating a response device.

5. A method as defined in claim 1, wherein activating a response device comprises controlling a household appliance.

6. A method as defined in claim 1, wherein the step of processing the health signal comprises receiving the signal at a multimedia server connected to the network, wherein the multimedia server determines if an emergency condition exists and, if an emergency condition does exist, transmits an emergency signal.

7. A method as defined in claim 1, wherein the receiving unit processes the health signal with information from an ambient environmental sensor.

8. A method as defined in claim 7, wherein the receiving unit processes the health signal by compensating for ambient temperature.

9. A method as defined in claim 7, wherein the receiving unit processes the health signal by compensating for an expected time of day fluctuation in the health signal.

10. A method as defined in claim 1, wherein:
the sensor unit comprises a temperature sensor that is attached to the individual and produces a temperature signal;
the temperature signal provides an indication of whether the individual is in a safe or emergency temperature condition; and
the step of producing a health signal comprises producing a temperature signal that indicates a temperature characteristic of the individual.

11. A method as defined in claim 1, wherein:
the sensor unit comprises a position detector that is attached to the individual and produces a health signal;
the health signal provides an indication of whether the individual is in an upright or a horizontal position; and
the step of producing a health signal comprises producing a digital position signal that indicates a position characteristic of the individual.

12. A method for monitoring health signs of an individual, the method comprising the steps of:
communicating a health signal from a sensor unit on the individual to a receiving unit; and
processing the health signal to determine if an emergency condition exists, wherein the sensor unit receives electrical power from a battery, battery power is determined by counting the number of health signal transmissions that have occurred since electrical power was last applied, and an indication of low battery power is provided when a predetermined number of transmissions have occurred.

13. A method as defined in claim 12, wherein the step of processing the health signal comprises receiving the signal at a multimedia server connected to a network, wherein the multimedia server determines if an emergency condition exists and, if an emergency condition does exist, transmits an emergency signal.

14. A method as defined in claim 12, wherein the receiving unit processes the health signal with information from an ambient environmental sensor.

15. A method as defined in claim 14, wherein the receiving unit processes the health signal by compensating for ambient temperature.

16. A method as defined in claim 14, wherein the receiving unit processes the health signal by compensating for an expected time of day fluctuation in the health signal.

17. A method as defined in claim 12, wherein operating electrical power is applied to the receiving unit in an initialization mode, said receiving unit determining if said receiving unit has received an identification signal from the sensor unit, and receiving a health signal only from a sensor unit having the received identification signal.

18. A method as defined in claim 17, further comprising:
halting the application of operating electrical power; and
repeating the steps of determining and receiving upon resuming the application of operating electrical power until a next identification signal is received.

19. A method as defined in claim 12, wherein the step of processing the health signal comprises responding to an emergency condition by activating a sensor unit.

20. A method as defined in claim 12, wherein the step of processing the health signal comprises responding to an emergency condition by activating a response device.

21. A method as defined in claim 20, wherein activating a response device comprises controlling a household appliance.

22. A method for continuously monitoring health signs of a patient over a period of time, the method comprising the steps of:
attaching a sensor unit to the patient;
producing a patient signal that indicates the current status of a patient health sign;
transmitting the patient signal to a receiving unit; and
displaying the patient's current health sign status at the receiving unit, wherein the sensor unit receives electrical power from a battery, battery power is determined by counting the number of signal transmissions that have occurred since electrical power was last applied, and an indication of low battery power is provided when a predetermined number of transmissions have occurred.

23. A method as defined in claim 22, further comprising the step of processing the patient signal to determine if the patient's current health sign status indicates an emergency condition and sending the patient signal over a network to a destination device.

24. A method as defined in claim 23, wherein the step of processing the patient signal comprises receiving the patient signal at a multimedia server connected to the network, wherein the multimedia server determines if an emergency condition exists and, if an emergency condition does exist, transmits an emergency signal over the network.

25. A method as defined in claim 23, wherein the receiving apparatus processes the patient signal with information from an ambient environmental sensor before providing the patient signal to the network.

26. A method as defined in claim 23, wherein the step of processing the patient signal comprises responding to the existence of an emergency condition by activating a sensor unit.

27. A method as defined in claim 23, wherein the step of processing the patient signal comprises responding to the existence of an emergency condition by controlling a household appliance.

28. A method as defined in claim 24, wherein the receiving unit processes the patient signal by compensating for ambient temperature.

29. A method as defined in claim 24, wherein the receiving unit processes the patient signal by compensating for an expected time of day fluctuation in the patient signal.

30. A system for monitoring health signs of an individual, said system comprising:
a sensor unit for producing a health signal that indicates at least one health sign of an individual, said sensor unit being located proximate to an individual; and
a receiving unit for receiving the health signal from said sensor unit over a wireless connection, said receiving unit including:
identification determination means for determining if said receiving unit has received an identification signal from said sensor unit;
means for allowing said receiving unit to receive the health signal from said sensor unit only after said receiving unit has received the identification signal from said sensor unit; and
emergency condition determination means for determining if an emergency condition exists based on the health signal received from said sensor unit.

31. A system as defined in claim 30, further comprising a server means for communicating an indication of an emergency condition from said receiving unit to a network when said emergency condition determination means determines that an emergency condition exists.

32. A system as defined in claim 30, wherein:
the sensor unit comprises a temperature sensor that is attached to the individual and produces a temperature signal;
the temperature signal provides an indication of whether the individual is in a safe or emergency temperature condition; and
the sensor unit produces a temperature signal that indicates a temperature characteristic of the individual.

33. A system as defined in claim 30, wherein the receiving unit processes the health signal with information from an ambient environmental sensor before providing the health signal to the network.

34. A system as defined in claim 3,wherein the receiving unit processes the health signal by compensating for ambient temperature.

35. A system as defined in claim 33, wherein the receiving unit processes the health signal by compensating for an expected time of day fluctuation in the health signal.

36. A system as defined in claim 30, wherein:
the sensor unit comprises a position detector that is attached to the individual and produces a health signal;
the health signal provides and indication of whether the individual is in an upright or a horizontal position; and
the sensor unit produces a position signal that indicates a position characteristic of the individual.

37. A system as defined in claim 30, wherein the receiving unit operates in the initialization mode at each application of electrical power, such that it registers a sensor unit identification code after each initialization.

38. A system as defined in claim 30, wherein the receiving unit processes the health signal by activating a sensor unit.

39. A system as defined in claim 30, wherein the system determines an emergency condition by considering multiple health signs signals.

40. A system as defined in claim 30, wherein the sensor unit is attached to a harness worn by the individual.

41. A system as defined in claim 30, wherein the sensor unit is attached to a garment worn by the individual.

42. A system as defined in claim 41, wherein the system indicates an emergency condition if the sensor unit is removed from the garment.

43. A system as defined in claim 30, wherein the receiving unit includes a display that provides a predetermined message in response to an emergency condition.

44. A system as defined in claim 43, wherein the predetermined message comprises an advertisement for a product that may be used to treat the emergency condition.

45. A system as defined in claim 30, wherein the receiving unit processes the provided signal by controlling a household device.

46. A system for monitoring health signs of an individual, said system comprising:
a sensor unit for producing a health signal that indicates at least one health sign of an individual, said sensor unit being located proximate to an individual; and
a receiving unit for receiving the health signal from said sensor unit over a wireless connection, said receiving unit including:
emergency condition determination means for determining if an emergency condition exists based on the health signal received from said sensor unit;
means for receiving electrical power from a battery;
means for determining battery power by counting the number of health signal transmissions that have been received by said receiving unit from said sensor unit since electrical power was last supplied to said receiving unit; and
means for providing an indication of low battery power when a predetermined number of health signal transmissions have occurred since electrical power was last supplied to said receiving unit.

47. A system as defined in claim 46, further comprising a server means for communicating an indication of an emergency condition from said receiving unit to a network when said emergency condition determination means determines that an emergency condition exists.

48. A system as defined in claim 46, wherein the receiving unit operates such that, upon the application of electrical power in an initialization mode, the receiving unit determines if the receiving unit has received an identification signal from the sensor unit, and receives a health signal only from a sensor unit having the received identification signal.

49. A system as defined in claim 48, wherein the receiving unit operates in the initialization mode at each application of electrical power, such that it registers a sensor unit identification code after each initialization.

50. A system as defined in claim 46, wherein the receiving unit processes the provided signal by activating a sensor.

51. A system as defined in claim 46, wherein the system determines an emergency condition by considering multiple health signs signals.

52. A system as defined in claim 46, wherein the sensor unit is attached to a harness worn by the individual.

53. A system as defined in claim 46, wherein the sensor unit is attached to a garment worn by the individual.

54. A system as defined in claim 46, wherein the receiving apparatus includes a display that provides a predetermined message in response to an emergency condition.

55. A system as defined in claim 46, wherein the receiving unit processes the provided signal by controlling a household device.

56. A system for monitoring health signs of an individual, said system comprising:
a receiving unit for receiving a health signal from a sensor unit over a wireless connection, said receiving unit including:
identification determination means for determining if said receiving unit has received an identification signal from said sensor unit;
means for allowing said receiving unit to receive the health signal from said sensor unit only after said receiving unit has received the identification signal from said sensor unit; and
emergency condition determination means for determining if an emergency condition exists based on the health signal received from said sensor unit.

57. A system as defined in claim 56, further comprising a server means for communicating an indication of an emergency condition from said receiving unit to a network when said emergency condition determination means determines that an emergency condition exists.

58. A system as defined in claim 56, wherein the health signal received by the receiving unit comprises a digital temperature signal that indicates a temperature characteristic of the individual in response to a temperature signal of the sensor unit.

59. A system as defined in claim 56, wherein the receiving unit processes the health signal with information from an ambient environmental sensor before providing the communicated health signal to the network.

60. A system as defined in claim 58, wherein the receiving unit processes the health signal by compensating for an expected time of day fluctuation in the health signal.

61. A system as defined in claim 56, wherein the receiving unit processes the health signal by compensating for ambient temperature.

62. A system as defined in claim 56, wherein the health signal received by the receiving unit comprises a position signal that indicates a position characteristic of the individual.

63. A system as defined in claim 56, wherein the receiving unit operates in the initialization mode at each application of electrical power, such that it registers a sensor unit identification code after each initialization.

64. A system for monitoring health signs of an individual, said system comprising:
a receiving unit for receiving a health signal from a sensor unit over a wireless connection, said receiving unit including:
emergency condition determination means for determining if an emergency condition exists based on the health signal received from said sensor unit;
means for receiving electrical power from a battery;
means for determining battery power by counting the number of health signal transmissions that have been received by said receiving unit from said sensor unit since electrical power was last supplied to said receiving unit; and
means for providing an indication of low battery power when a predetermined number of health signal transmissions have occurred since electrical power was last supplied to said receiving unit.

65. A system as defined in claim 64, further comprising a server means for communicating an indication of an emergency condition from said receiving unit to a network when said emergency condition determination means determines that an emergency condition exists.

66. A system as defined in claim 64, wherein the receiving unit operates such that, upon the application of electrical power in an initialization mode, the receiving unit determines if the receiving unit has received an identification signal from the sensor unit, and receives a health signal only from a sensor unit having the received identification signal.

67. A system as defined in claim 66, wherein the receiving unit operates in the initialization mode at each application of electrical power, such that it registers a sensor unit identification code after each initialization.

68. A system for monitoring health signs of an individual, said system comprising:
a first sensor unit for producing a health signal that indicates at least one health sign of an individual, said sensor unit being located proximate to an individual; and
a receiving unit for receiving the health signal from said sensor unit over a wireless connection, said receiving unit including:
means for determining the format of said health signal from said first sensor unit; and
emergency condition determination means for determining if an emergency condition exists based on the health signal received from said sensor unit.

69. A system as defined in claim 68, further comprising a server means for communicating an indication of an emergency condition from said receiving unit to a network when said emergency condition determination means determines that an emergency condition exists.

70. A system as defined in claim 68, wherein if said receiving unit determines that said first sensor unit is transmitting a health signal in a format that can be processed by said receiving unit, said receiving unit processes said health signal.

71. A system as defined in claim 68, wherein if said receiving unit determines that said first sensor unit is transmitting a health signal in a format that can not be processed by said receiving unit, said receiving unit sends a request to a network computer for the receiving unit software to be updated to allow said health signal to be processed.

72. A system as defined in claim 68, wherein said first sensor unit includes a first transceiver that transmits the health signal from the first sensor unit to the receiving unit.

73. A system as defined in claim 68, wherein said receiving unit comprises a display.

74. A system as defined in claim 73, wherein said health signal is processed by said receiving unit and processing said health signal includes displaying data representative of said health signal on said display.

75. A system as defined in claim 68, wherein said first sensor unit is replaced with a second sensor unit that has a health signal format different from a health signal format of said first sensor unit.

76. A system as defined in claim 75, wherein if said receiving unit determines that said second sensor unit is transmitting a signal in a format that can be processed by said receiving unit, said receiving unit processes said health signal.

77. A system as defined in claim 76, wherein if said receiving unit determines that said second sensor unit is transmitting a signal in a format that can not be processed by said receiving unit, said receiving unit sends a request to a network computer for the receiving unit software to be updated to allow said health signal to be processed.

78. A system as defined in claim 77, wherein said second sensor unit includes a second transceiver that transmits the health signal from the second sensor unit to the receiving unit.

79. A method for monitoring health signs of an individual, the method comprising:
detecting at least one health sign characteristic of the individual with a sensor unit that is located proximate to the individual;
producing a health signal from the sensor unit that indicates at least one health sign of the individual;
communicating the health signal from the individual to a receiving unit over a wireless connection;
extracting at least one health factor from an independent data source; and
processing the health signal and the extracted health factor to determine if an emergency condition exists.

80. A method as defined in claim 79, wherein said health factor comprises a pollen count.

81. A method as defined in claim 79, wherein said health factor comprises an outdoor air temperature.

82. A method as defined in claim 79, wherein said independent data source comprises the Internet.

83. A method as defined in claim 79, wherein said independent data source comprises a stored database.

84. A method as defined in claim 79, wherein said independent data source comprises a pill dispenser.

85. A method as defined in claim 79, wherein said independent data source comprises an invoice.
